(19) United States
(12) Patent Application Publication
(10) Pub. No.: US 2023/0255499 A1
(43) Pub. Date: Aug. 17, 2023

(54) APPARATUS AND METHOD FOR ESTIMATING BODY TEMPERATURE

(71) Applicants: SAMSUNG ELECTRONICS CO., LTD., Suwon-si (KR); SEOUL NATIONAL UNIVERSITY R&DB FOUNDATION, Seoul (KR)

(72) Inventors: So Young LEE, Suwon-si (KR); Joo-Young LEE, Seoul (KR); Dahee JUNG, Seoul (KR); Bok Soon KWON, Seoul (KR); Sang Kyu KIM, Yongin-si (KR); Ho Taik LEE, Yongin-si (KR)

(73) Assignees: SAMSUNG ELECTRONICS CO., LTD., Suwon-si (KR); SEOUL NATIONAL UNIVERSITY R&DB FOUNDATION, Seoul (KR)

(21) Appl. No.: 17/953,050

(22) Filed: Sep. 26, 2022

(30) Foreign Application Priority Data
Feb. 16, 2022 (KR) 10-2022-0020330

(51) Int. Cl. A61B 5/0205; A61B 5/026; A61B 5/0295; A61B 5/00; A61B 5/024

(52) U.S. Cl. CPC A61B 5/02055; A61B 5/0261; A61B 5/0295; A61B 5/4866; A61B 5/7278; A61B 5/7275; A61B 5/02416; A61B 2560/0223; A61B 5/681

(57) ABSTRACT

An apparatus for estimating body temperature, may include: a photoplethysmogram (PPG) sensor configured to measure a PPG signal by emitting light onto an object and by detecting light scattered or reflected from the object; and a processor configured to estimate a cutaneous blood flow and an energy metabolism of the object based on the PPG signal, estimate a core body temperature of the object based on the cutaneous blood flow and the energy metabolism, and provide notification information on a risk of abnormal body temperature based on the core body temperature.

### CROSS-REFERENCE TO RELATED APPLICATION(S)

This application claims priority from Korean Patent Application No. 10-2022-0020330, filed on Feb. 16, 2022, in the Korean Intellectual Property Office, the disclosure of which is incorporated herein by reference in its entirety.

### BACKGROUND

### 1. Field

Apparatuses and methods consistent with example embodiments relate to estimating core body temperature using a sensor and providing notification information on a risk of abnormal body temperature.

### 2. Description of the Related Art

Generally, body temperature is one of four vital signs and has very important clinical significance. Failure to control body temperature may put people's lives at risk. Particularly, people working in hot environments or doing outdoor activities during a heatwave in the summertime or people enjoying mountain sports and activities in low temperature environments may be at risk of losing their lives. A risk index based on air temperature and humidity is used to prevent accidents and deaths caused by abnormal body temperature, and workers exposed to health risks generally determine their activities based on the risk index. However, the risk index may not sufficiently reflect each individual's ability to adapt to heat or cold or physical activities. Accordingly, there is a need for a method of directly monitoring core body temperature of each individual and for providing notification of risk of abnormal body temperature based on the core body temperature.

### SUMMARY

According to an aspect of the disclosure, an apparatus for estimating body temperature may include: a photoplethysmogram (PPG) sensor configured to measure a PPG signal by emitting light onto an object and by detecting light scattered or reflected from the object; and a processor configured to estimate a cutaneous blood flow and an energy metabolism of the object based on the PPG signal, estimate a core body temperature of the object based on the cutaneous blood flow and the energy metabolism, and provide notification information on a risk of abnormal body temperature based on the core body temperature.

The processor may be further configured to obtain a reference cutaneous blood flow and a reference energy metabolism based on a calibration PPG signal measured by the PPG sensor at a calibration time, determine weights to be applied to the reference cutaneous blood flow and the reference energy metabolism, respectively, and generate a core body temperature estimation model for estimating the core body temperature based on a weighted sum of the reference cutaneous blood flow and the reference energy metabolism.

The processor may be further configured to determine a first weight for the cutaneous blood flow based on heat generated from an external heat source, and determine a second weight for the energy metabolism based on an internally generated heat.

The processor may be configured to extract a feature from the PPG signal, and estimate the cutaneous blood flow based on the extracted feature by using a model that defines a correlation between the feature and the cutaneous blood flow.

The processor may be further configured to obtain a heart rate from the PPG signal, and estimate the energy metabolism based on the heart rate.

The processor may be further configured to estimate the core body temperature of the object by applying the cutaneous blood flow and the energy metabolism to a predetermined core body temperature estimation model.

The apparatus may further include an output interface, wherein in response to the estimated core body temperature falling outside a predetermined threshold range, the processor may be further configured to provide the notification information on the risk of abnormal body temperature through the output interface.

In response to the estimated core body temperature falling outside a predetermined temperature range, the processor may be further configured to provide a text message or a voice message that recommends stopping outdoor activities.

The processor may be further configured to estimate the core body temperature of the object based further on at least one of a user's motion and temperature while the PPG signal is measured.

According to another aspect of the disclosure, a method of estimating body temperature may include: measuring a photoplethysmogram (PPG) signal by emitting light onto an object and by detecting light scattered or reflected from the object; estimating cutaneous blood flow and energy metabolism based on the PPG signal; estimating core body temperature of the object based on the cutaneous blood flow and the energy metabolism; and providing notification information on a risk of abnormal body temperature based on the estimated core body temperature.

The method may further include obtaining a reference cutaneous blood flow and a reference energy metabolism based on a calibration PPG signal at a calibration time, determining weights to be applied to the reference cutaneous blood flow and the reference energy metabolism, respectively, and generating a core body temperature estimation model for estimating the core body temperature based on a weighted sum of the reference cutaneous blood flow and the reference energy metabolism.

The generating of the core body temperature estimation model may include determining a first weight for the cutaneous blood flow based on heat generated from an external heat source, and determining a second weight for the energy metabolism based on an internally generated heat.

The estimating of the cutaneous blood flow may include extracting a feature from the PPG signal, and estimating the cutaneous blood flow based on the extracted feature by using a model that defines a correlation between the feature and the cutaneous blood flow.

The estimating of the energy metabolism may include obtaining a heart rate from the PPG signal, and estimating the energy metabolism based on the heart rate.

The estimating of the core body temperature of the object may include estimating the core body temperature of the object by applying the cutaneous blood flow and the energy metabolism to a predetermined core body temperature estimation model.

The providing of the notification information on the risk of abnormal body temperature may include, in response to the estimated core body temperature falling outside a predetermined threshold range, providing the notification information on the risk of abnormal body temperature through an output interface.

The method may further include, in response to the core body temperature falling outside a predetermined threshold range, providing a text message or a voice message that recommends stopping outdoor activities.

The estimating of the core body temperature of the object may include estimating the core body temperature of the object based further on at least one of a user's motion and temperature while the PPG signal is measured.

According to another aspect of the disclosure, an electronic device may include: a display configured to provide a user interface to receive health profile information; a PPG sensor configured to measure a PPG signal by emitting light onto a user and by detecting light scattered or reflected from the user; and a processor configured to estimate a cutaneous blood flow and an energy metabolism of the user based on the PPG signal, estimate a core body temperature of the user based on the cutaneous blood flow and the energy metabolism, and provide notification information on a risk of abnormal body temperature based on the core body temperature, and the health profile information.

The health profile information may include a weight, a height, and an age of the user, and one of a plurality of temperature measurement sites in a body of the user which is selected by the user.

### BRIEF DESCRIPTION OF THE DRAWINGS

The above and/or other aspects will be more apparent by describing certain example embodiments, with reference to the accompanying drawings, in which:

FIG. 1 is a block diagram illustrating an apparatus for estimating body temperature according to embodiments of the present disclosure;

FIG. 2 is a diagram illustrating an example of estimating cutaneous blood flow based on a feature extracted from a PPG signal, according to embodiments of the present disclosure;

FIGS. 3 and 4 are diagrams illustrating an example of providing notification information on a risk of abnormal body temperature in a wristwatch-type wearable device including an apparatus for estimating body temperature according to embodiments of the present disclosure;

FIG. 5 is a block diagram illustrating an apparatus for estimating body temperature according to embodiments of the present disclosure;

FIG. 6 is a flowchart illustrating a method of estimating body temperature according to embodiments of the present disclosure.

FIGS. 7 to 10 are diagrams illustrating examples of structures of electronic devices including an apparatus for estimating body temperature, according to embodiments of the present disclosure.

### DETAILED DESCRIPTION

Example embodiments are described in greater detail below with reference to the accompanying drawings.

In the following description, like drawing reference numerals are used for like elements, even in different drawings. The matters defined in the description, such as detailed construction and elements, are provided to assist in a comprehensive understanding of the example embodiments. However, it is apparent that the example embodiments can be practiced without those specifically defined matters. Also, well-known functions or constructions are not described in detail since they would obscure the description with unnecessary detail.

It will be understood that, although the terms first, second, etc. may be used herein to describe various elements, these elements should not be limited by these terms. These terms are only used to distinguish one element from another. Also, the singular forms are intended to include the plural forms as well, unless the context clearly indicates otherwise. It will be further understood that when an element is referred to as “comprising” another element, the element is intended not to exclude one or more other elements, but to further include one or more other elements, unless explicitly described to the contrary. In the following description, terms such as “unit” and “module” indicate a unit for processing at least one function or operation and they may be implemented by using hardware, software, or a combination thereof.

Expressions such as “at least one of,” when preceding a list of elements, modify the entire list of elements and do not modify the individual elements of the list. For example, the expression, “at least one of a, b, and c,” should be understood as including only a, only b, only c, both a and b, both a and c, both b and c, all of a, b, and c, or any variations of the aforementioned examples.

Hereinafter, embodiments of an apparatus and method for estimating body temperature will be described in detail with reference to the accompanying drawings.

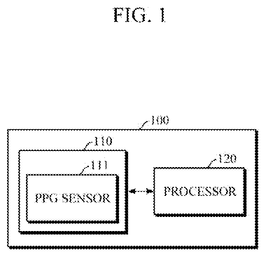

FIG. 1 is a block diagram illustrating an apparatus for estimating body temperature according to an embodiment of the present disclosure.

An apparatus 100 for estimating body temperature may be mounted in various devices, such as a portable wearable device, a smart device, and the like. For example, examples of the various devices may include a wearable device such as a smart watch worn on the wrist, a smart band-type wearable device, a headphone-type wearable device, a headband-type wearable device, an arm band-type wearable device, a chest band-type wearable device, etc., or a mobile device such as a smartphone, a tablet PC, etc., but are not limited thereto.

Referring to FIG. 1, the apparatus 100 for estimating body temperature includes a sensor 110 and a processor 120.

The sensor 110 may obtain data for estimating body temperature from an object, and the processor 120 may estimate body temperature of the object by using the data obtained by the sensor 110. The processor 120 and the sensor 110 may be electrically connected to each other and upon receiving a request for estimating body temperature, the processor 120 may control the sensor 110. The object may be a body part which may come into contact with the sensor 110, and may be, for example, a body part at which pulse waves may be easily measured. For example, the object may be fingers, earlobes, ankles, etc., where blood vessels are densely distributed in the human body, but is not limited thereto, and may be an area on the wrist that is adjacent to the radial artery and the upper part of the wrist where venous blood or capillary blood passes, and a peripheral part of the human body, such as toes and the like.

The sensor 110 may include a photoplethysmogram (PPG) sensor 111. The PPG sensor 111 may measure a PPG signal by emitting light onto the object and by detecting light scattered or reflected from the object. The PPG sensor 110 may have a plurality of channels, in which each channel may include one or more light sources for emitting light of one or more wavelengths onto the object and may be disposed at different distances so as to measure the PPG signal at different positions of the object. In addition, each channel of the PPG sensor 111 may include one or more detectors for detecting light returning by being scattered or reflected from or transmitted into body tissue, such as the object's skin surface, blood vessels, etc., after light is emitted by the light source. The detector may include a photo diode, a photo transistor, an image sensor (e.g., complementary metal-oxide semiconductor (CMOS) image sensor), etc., but is not limited thereto.

The processor 120 may estimate cutaneous blood flow and energy metabolism based on the PPG signal acquired by the PPG sensor 111, and may estimate core body temperature of the object based on the estimated cutaneous blood flow and energy metabolism.

For example, based on the PPG signal acquired by the PPG sensor 111, the processor 120 may estimate the cutaneous blood flow. For example, the processor 120 may extract a feature from the PPG signal, and may estimate the cutaneous blood flow based on the extracted feature by using a model that defines a correlation between the feature and the cutaneous blood flow.

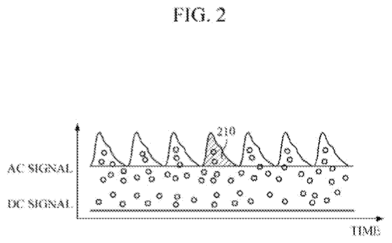

FIG. 2 is a diagram illustrating an example of estimating cutaneous blood flow based on a feature extracted from a PPG signal, according to an embodiment of the present disclosure.

The PPG signal is composed of a DC signal and an AC signal, in which the DC signal is a constant signal for bone and muscle, and the AC signal is a signal that changes according to a blood flow in the artery that is pumped from the heart. The DC signal and the AC signal may correspond to a nonpulsatile component and a pulsatile component of the PPG signal. Referring to FIG. 2, an upper portion of the graph shows the AC signal that changes according to a pulse wave, and a lower portion of the graph shows the constant DC signal. The AC signal may indicate whether the volume of arterial blood increases or decreases, thereby indirectly reflecting a change in the blood flow. Accordingly, the processor 120 may extract, as a feature of the PPG signal, an area under curve (AUC) which is an area 210 of an AC component the PPG signal, and may estimate the cutaneous blood flow by using the extracted AUC. In this case, the processor 120 may obtain the cutaneous blood flow based on the extracted AUC by using a blood flow estimation model that defines a correlation between the AUC and the cutaneous blood flow.

In another example, the processor 12 may extract, as a feature of the PPG signal, a Perfusion Index that indicates a ratio of the AC signal to the DC signal of the PPG signal, and may estimate the cutaneous blood flow by using the obtained Perfusion Index. For example, the processor 120 may obtain the cutaneous blood flow based on the obtained Perfusion Index by using a blood flow estimation model that defines a correlation between the Perfusion Index and the cutaneous blood flow.

PI(Perfusion Index)=AC/DC [Equation 1]

However, the feature is not limited thereto, and as other features of the PPG signal for estimating the cutaneous blood flow, the processor 120 may extract a characteristic point, such as a time point associated with a propagation wave component of the PPG signal and an amplitude corresponding to the time point, a time point associated with a reflection wave component of the PPG signal and an amplitude corresponding to the time point. In this case, the processor 120 may extract the characteristic points associated with the propagation wave component and the reflection wave component based on a second derivative signal of the PPG signal. For example, the processor 120 may extract a time point corresponding to a first local minimum point of the second derivative signal as the time point associated with the propagation wave component, and time points corresponding to second and third local minimum points as time points associated with the reflection wave component. In addition, the processor 120 may extract, as characteristic points, a time point and/or an amplitude in a predetermined region of the PPG signal, e.g., a time point and/or an amplitude at a maximum amplitude point, an internally dividing point between a time point corresponding to the maximum amplitude point and the time point associated with the propagation wave component, and/or an amplitude corresponding to the time point. In this case, the internally dividing point may be a midpoint between the two time points or a point at which the time points are internally divided in a predetermined ratio.

Further, the processor 120 may obtain information of energy metabolism based on the PPG signal acquired by the PPG sensor 111. For example, the processor 120 may obtain heart rate (HR) from the PPG signal, and may estimate energy metabolism based on the obtained HR.

First, the processor 120 may obtain the HR by analyzing a period of the PPG signal which is measured continuously over time. For example, upon acquiring a PPG signal having 100 periods per minute, the processor 120 may set the heart rate to 100.

Then, the processor 120 may estimate the energy metabolism based on the obtained heart rate. In particular, the processor 120 may continuously output an estimation of the energy metabolism which is updated in real time as the PPG signal and the heart rate are continuously measured over time. For example, the processor 120 may estimate the energy metabolism MHR based on the obtained heart rate (HR) by using the following Equations 2 to 5.

$${M}_{HR} = {M}_{0} + \frac{( MWC - {M}_{0} )}{( {HR}_{max} - {HR}_{0} )} \times ( HR - {HR}_{0} ) \qquad [ Equation 2 ]$$

$$MWC = ( 19.45 - 0.133 \times age ) \times LBM \qquad [ Equation 3 ]$$

$$LBM = ( 1.08 - \frac{{W}_{b}}{80 \times {H}_{b}^{2}} ) \times {W}_{b} \qquad [ Equation 4 ]$$

$${HR}_{max} = 208 - 0.7 \times age . \qquad [ Equation 5 ]$$

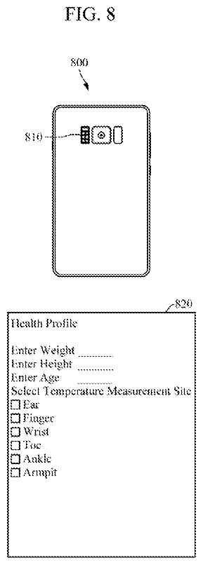

Herein, M₀ denotes a resting metabolic rate, e.g., metabolism at rest or basal metabolism, and an adult man generally has a metabolic rate of 60 W/m²; HR₀ denotes a resting heart rate, e.g., heart rate at rest; a maximum work capacity in watts (MWC) denotes metabolism at maximum work capacity; HRₘₐₓ denotes a maximum heart rate; lean body mass (LBM) denotes body weight without fat; Hb denotes height; Wb denotes weight; and age denotes age. In this case, previously measured and stored values may be used as M₀ and HR₀, and the processor 120 may obtain the height, weight, and age from a user through a display device using an interface (e.g., see a user interface 820 illustrated in FIG. 8), or may use previously stored values.

In addition, the apparatus 100 for estimating body temperature may further include a sensor for estimating oxygen consumption, and based on oxygen consumption of an object which is estimated by the sensor, the processor 120 may estimate energy metabolism M_{VO2} by using the following Equation 6.

M_{VO2}=VO₂×60×5.68 [Equation 6]

Herein, VO₂ denotes the oxygen consumption; and 5.68 denotes standard energy equivalent of 5.68 W/(1/h) according to ISO 8996.

Subsequently, the processor 120 may estimate core body temperature of the object by applying the estimated cutaneous blood flow and energy metabolism to a predetermined core body temperature estimation model. In this case, the predetermined core body temperature estimation model may be represented by the following Equation 7.

T_{c}=α×SBP^{a}+β×EE^{b} [Equation 7]

Herein, T_{c} denotes the core body temperature; SBF denotes the cutaneous blood flow; EE denotes the energy consumption; a and b denote predetermined coefficients; and α and β denote weights of the cutaneous blood flow and the energy metabolism, respectively. The weights α and β will be described in detail later in a process of generating a core body temperature estimation model.

Next, the processor 120 may provide notification information on a risk of abnormal body temperature. The processor 120 may determine a measured body temperature as an abnormal body temperature when the measured body temperature is outside a predetermined temperature range between a predetermined lower threshold and a predetermined upper threshold. If the abnormal body temperature is less than the predetermined upper threshold, the processor 120 may determine the temperature as a high body temperature, and if the abnormal body temperature is less than or equal to the predetermined lower threshold, the processor 120 may determine the temperature as a low body temperature. For example, the apparatus 100 for estimating body temperature may further include an output interface, and if the estimated core body temperature is outside the predetermined temperature range, the processor 120 may provide notification information on a risk of abnormal body temperature through the output interface. For example, upon determining that the estimated core body temperature is abnormal, the processor 120 may provide notification information to stop outdoor activities by using a text message or a voice message. The processor 120 may set a temperature range for determining an abnormal body temperature differently according to a temperature measurement site (e.g., an ear, a finger, a wrist, a toe, an ankle, or an armpit), which is selected by a user via a user interface (e.g., a user interface 820 illustrated in FIG. 8).

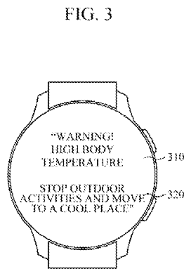

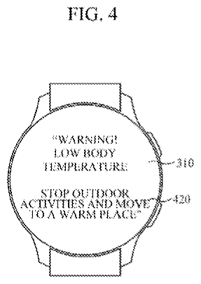

FIGS. 3 and 4 are diagrams illustrating an example of providing notification information on a risk of abnormal body temperature in a wristwatch-type wearable device including the apparatus 100 for estimating body temperature according to an embodiment of the present disclosure. For example, referring to FIG. 3, if the estimated core body temperature is in a range greater than or equal to a predetermined threshold range, the processor 120 may output a text message 320, “WARNING! HIGH BODY TEMPERATURE” and “stop outdoor activities and move to a cool place,” through a display device 310. In addition, referring to FIG. 4, if the estimated core body temperature is in a range less than or equal to the predetermined threshold range, the processor 120 may output a text message 420, “WARNING!LOW BODY TEMPERATURE” and “stop outdoor activities and move to a warm place,” through the display device 310. In this case, the processor 120 may output a voice message for guiding a user to stop outdoor activities, or may output the text message and the voice message at the same time. In addition, the processor 120 may output a warning alarm along with the text message and the voice message through the output interface. If the estimated core body temperature remains outside the predetermined temperature range even when a predetermined period of time elapses after providing the notification information on the risk of abnormal body temperature, the processor 120 may automatically call 119 safety center by communicating with an electronic device (e.g., smartphone). However, the method of providing the notification information on the risk of the abnormal body temperature is not limited thereto.

Generally, the core body temperature may be measured at a plurality of body parts, e.g., esophagus, stomach, rectum, eardrum, armpit, etc., and the measured values may slightly vary depending on the body parts. Accordingly, a threshold range for determining abnormal body temperature may be set differently for each reference body part. For example, in the case where the apparatus 100 for estimating body temperature is mounted in a wristwatch-type wearable device or a chest band-type wearable device, the armpit relatively close to the device may be determined to be a reference body part for determining a threshold range. In this case, for example, the apparatus 100 for estimating body temperature may determine in advance a range of 35° C. to 40° C. to be a threshold range based on the armpit. In another example, in the case where the apparatus 100 for estimating body temperature is mounted in a earbuds-type wearable device, the eardrum relatively close to the device may be determined to be a reference body part for determining a threshold range. In this case, for example, the apparatus 100 for estimating body temperature may determine in advance a range of 35° C. to 39.5° C. to be a threshold range based on the eardrum. In yet another example, in the case where the apparatus 100 for estimating body temperature is mounted in a wristwatch-type wearable device, the apparatus 100 for estimating body temperature may measure in advance a difference in core body temperature between the armpit and the wrist by preprocessing, and may set a threshold range for the wrist by correcting a threshold range for the armpit based on the measured difference, and may directly use the set threshold range for the wrist. The information of a temperature measurement site in the body of a user may be obtained via a user interface (e.g., a user interface 820 in FIG. 8). However, the method of determining the threshold range is not limited thereto.

For example, the apparatus 100 for estimating body temperature may further include a motion sensor and/or a temperature sensor, and during measurement of the PPG signal, the processor 120 may estimate core body temperature of the object based further on a user's motion information and/or temperature. For example, the processor 120 may reflect the energy metabolism with improved accuracy by using motion data obtained by the motion sensor, and may increase the accuracy of estimating core body temperature by further adding skin temperature, sensed by the temperature sensor, as a parameter of the core body temperature estimation model.

In addition, the processor 120 may generate in advance the core body temperature estimation model in order to increase the accuracy of estimating body temperature. For example, the processor 120 may obtain cutaneous blood flow and energy metabolism based on a calibration PPG signal measured by the PPG sensor at a calibration time, may determine weights to be applied to the obtained cutaneous blood flow and energy metabolism, respectively, and may generate the core body temperature estimation model for estimating the core body temperature by linearly combining the weighted values.

The above Equation 7 is the generated core body temperature estimation model, in which α and β denote weights of the cutaneous blood flow and the energy metabolism, respectively. In this case, the processor 120 may determine the weight α for the cutaneous blood flow based on heat generated from passive heating which is provided by an external source, and may determine the weight β based on heat generated from active heating which is provided internally.

The weight α for the cutaneous blood flow and the weight β for the energy metabolism may be determined by the following Equations 8 to 10.

T_{c}−T₀=α(SBF−SBF₀)^{a}+β(EE−EE₀)^{b} [Equation 8]

Herein, T_{c} denotes the core body temperature at the calibration time; SBF denotes the cutaneous blood flow at the calibration time; EE denotes the energy metabolism at the calibration time; a and b denote predetermined coefficients; T₀, SBF₀, and EE₀ denote initial values of the core body temperature, cutaneous blood flow, and energy metabolism with no heat transferred from the outside source or generated internally.

First, in the case of passive heating using heat provided by an external source, e.g., local heating of skin by using a heating pad, or in the case where a user takes a foot bath, the core body temperature increases from 36.5° C. to 42° C. (in this case, T₀ is 36.5° C., and T_{c} is 42° C.). In this case, there is relatively less active activity compared to the change in cutaneous blood flow, such that a variation EE−EE₀ in the energy metabolism of Equation 8 is considered zero, and the weight α for the cutaneous blood flow may be represented by the following Equation 9.

$$α = \frac{( Tc - {T}_{0} )}{{( SBF - {SBF}_{0} )}^{a}} \qquad [ Equation 9 ]$$

In the case of active heating using heat generated internally by active activity, e.g., running with 50% of a maximum VO₂, the weight β for the energy consumption may be represented by the following Equation 10. Here, a weight determined during the passive heating may be used as the weight α.

$$β = \frac{( Tc - {T}_{0} ) - {α ( SBF - {SBF}_{0} )}^{a}}{{( EE - {EE}_{0} )}^{b}} \qquad [ Equation 10 ]$$

The above embodiment shows an example of generating the core body temperature estimation model by obtaining the weight α for the cutaneous blood flow and the weight β for the energy consumption by using a heating method, i.e., passive heating and active heating. By contrast, the core body temperature estimation model may also be generated by obtaining the weight α for the cutaneous blood flow and the weight β for the energy consumption by using a cooling method, i.e., passing cooling (e.g., local cooling of skin using a cooling pad) and active cooling (e.g., reducing movement so as to decrease the VO₂ value).

Regarding a method of generating the core body temperature estimation model, for example, the core body temperature estimation model may be generated by obtaining the weight α for the cutaneous blood flow and the weight β for the energy consumption according to a reference position (wrist in the case of a wearable watch, and eardrum in the case of earbuds) which is determined based on the type of wearable device used when the core body temperature estimation model is generated. In this case, a threshold range may be set differently for each reference position as described above, and a predetermined threshold range may also be used.

By applying the obtained weight α for the cutaneous blood flow and the obtained weight β for the energy consumption to the core body temperature estimation model, body temperature change may be reflected based on each individual's characteristics, such that the accuracy of estimating body temperature may be improved, and thus information as to whether each individual is at risk of abnormal body temperature may be reflected accurately.

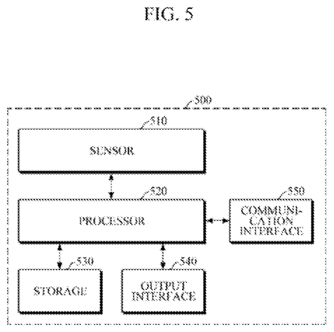

FIG. 5 is a block diagram illustrating an apparatus for estimating body temperature according to another embodiment of the present disclosure.

Referring to FIG. 5, an apparatus 500 for estimating body temperature includes a sensor 510, a processor 520, a storage 530, an output interface 540, and a communication interface 550. In this case, the sensor 510 and the processor 520 are the same as the sensor 110 and the processor 120 in the embodiment of FIG. 1, such that a detailed description thereof will be omitted.

The storage 530 may store information related to estimating core body temperature. For example, the storage 530 may store the PPG signal acquired by the sensor 510, processing results of the processor 520, such as the cutaneous blood flow and the energy metabolism, and the like.

The storage 530 may include at least one storage medium of a flash memory type memory, a hard disk type memory, a multimedia card micro type memory, a card type memory (e.g., an SD memory, an XD memory, etc.), a Random Access Memory (RAM), a Static Random Access Memory (SRAM), a Read Only Memory (ROM), an Electrically Erasable Programmable Read Only Memory (EEPROM), a Programmable Read Only Memory (PROM), a magnetic memory, a magnetic disk, and an optical disk, and the like, but is not limited thereto.

The output interface 540 may provide the processing results of the processor 520 for a user. In this case, the output interface 540 may display an estimated body temperature value along with a predetermined threshold range on a display. In this case, if the estimated body temperature value falls outside the threshold range, the output interface 540 may provide the user with warning information by changing color, line thickness, etc., so that the user may easily recognize the abnormal value. Further, along with or without the visual output displayed on the display, the output interface 540 may provide notification information on a risk of abnormal body temperature for the user in a non-visual manner by voice, vibrations, tactile sensation, and the like using an audio output module such as a speaker, or a haptic module and the like.

The communication interface 550 may communicate with an external device to transmit and receive various data, related to estimating the core body temperature, to and from the external device. In this case, the external device may include an information processing device such as a smartphone, a tablet PC, a desktop computer, a laptop computer, and the like. For example, the communication interface 550 may transmit a body temperature estimation result to the external device, such as a user's smartphone and the like, so that the user may manage and monitor the body temperature estimation result by using a device having a relatively high performance, and may provide notification information on the risk of abnormal body temperature with accuracy based on the estimation result.

In this case, the communication interface 550 may communicate with the external device by using various wired or wireless communication techniques, such as Bluetooth communication, Bluetooth Low Energy (BLE) communication, Near Field Communication (NFC), WLAN communication, Zigbee communication, Infrared Data Association (IrDA) communication, Wi-Fi Direct (WFD) communication, Ultra-Wideband (UWB) communication, Ant+ communication, WIFI communication, Radio Frequency Identification (RFID) communication, 3G, 4G, and 5G communications, and the like. However, this is merely exemplary and is not intended to be limiting.

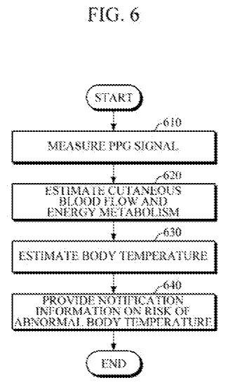

FIG. 6 is a flowchart illustrating a method of estimating body temperature according to an embodiment of the present disclosure.

The method of FIG. 6 is an example of a method of estimating body temperature performed by the apparatuses 100 and 500 for estimating body temperature according to the embodiments of FIG. 1 or FIG. 5, which are described above in detail, and thus will be briefly described below in order to avoid redundancy.

Referring to FIG. 6, the apparatus for estimating body temperature may first measure a PPG signal by using a photoplethysmogram (PPG) sensor which emits light onto an object and detects light scattered or reflected from the object in operation 610.

Then, the apparatus for estimating body temperature may estimate cutaneous blood flow and energy metabolism based on the PPG signal in operation 620.

The apparatus for estimating body temperature may extract a feature from the PPG signal, and may estimate the cutaneous blood flow based on the extracted feature by using a model that defines a correlation between the feature and the cutaneous blood flow. For example, the apparatus for estimating body temperature may extract, as a feature of the PPG signal, an area under curve (AUC) which is an area of the AC component of the PPG signal, and may estimate the cutaneous blood flow by using the extracted AUC. In this case, the apparatus for estimating body temperature may obtain the cutaneous blood flow based on the extracted AUC by using a blood flow estimation model which defines a correlation between the AUC and the cutaneous blood flow.

The apparatus for estimating body temperature may obtain heart rate from the PPG signal, and may estimate the energy metabolism based on the obtained heart rate. For example, the apparatus for estimating body temperature may obtain the heart rate by analyzing periods of the PPG signal which is measured continuously over time, and may estimate the energy metabolism based on the obtained heart rate. For example, by using the obtained heart rate (HR), the apparatus for estimating body temperature may estimate the energy metabolism MHR by using Equations 2 to 5.

Subsequently, the apparatus for estimating body temperature may estimate core body temperature of the object based on the estimated cutaneous blood flow and energy metabolism in operation 630. For example, the apparatus for estimating body temperature may estimate the core body temperature of the object by applying the cutaneous blood flow and the energy metabolism to a predetermined core body temperature estimation model. In this case, the apparatus for estimating body temperature may estimate the core body temperature of the object based further on at least one of a user's motion, measured by the motion sensor, and temperature measured by the temperature sensor while the PPG signal is measured.

Next, the apparatus for estimating body temperature may provide notification information on a risk of abnormal body temperature based on the estimated core body temperature in operation 640. For example, if the estimated core body temperature is higher than or equal to a predetermined threshold value, the apparatus for estimating body temperature may provide the notification information on the risk of abnormal body temperature through the output interface. In this case, the apparatus for estimating body temperature may output the notification information on the risk of abnormal body temperature as a text message, “WARNING! HIGH BODY TEMPERATURE” and “stop outdoor activities and move to a cool place,” or a text message, “WARNING! LOW BODY TEMPERATURE” and “stop outdoor activities and move to a warm place,” through the display device. In this case, the apparatus for estimating body temperature may also output a voice message for guiding a user to stop outdoor activities, or may output the text message and the voice message at the same time. In addition, the apparatus for estimating body temperature may output a warning alarm along with the text message and the voice message through the output interface. Even when a predetermined period of time elapses after the notification information on the risk of abnormal body temperature is provided, if the estimated core body temperature is maintained at a value greater than or equal to a threshold value, the apparatus for estimating body temperature may directly call 119 safety center by communicating with another electronic device.

A method of estimating body temperature according to another embodiment of the present disclosure may further include: obtaining cutaneous blood flow and energy metabolism based on a calibration PPG signal measured by the PPG sensor at a calibration time; determining weights to be applied to the obtained cutaneous blood flow and energy metabolism, respectively; and generating a core body temperature estimation model by linearly combining the weighted values. In this case, the apparatus for estimating body temperature may determine a weight for a variation in the cutaneous blood flow based on heat generated from passive heating which is provided by an external source, and may determine a weight for a variation in the energy metabolism based on heat generated from active heating which is provided internally.

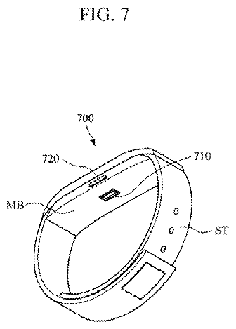

FIGS. 7 to 10 are diagrams illustrating examples of structures of electronic devices including the apparatuses 100 and 500 for estimating body temperature. Examples of the electronic device may include not only a smartphone, but also a smart watch, a smart band, smart glasses, a smart necklace, and an ear-wearable device, but the electronic device is not limited thereto.

Referring to FIG. 7, the electronic device may be implemented as a smart watch-type wearable device 700 which includes a main body MB and a wrist strap ST.

The main body MB may be formed in various shapes, and a battery may be embedded in the main body MB and/or the strap ST to supply power to various components of the wearable device. The strap ST may be connected to both ends of the main body to allow the main body to be worn on a user's wrist, and may be flexible so as to be wrapped around the user's wrist. The strap ST may be composed of a first strap and a second strap which are separated from each other. One ends of the first strap and the second strap are connected to both sides of the main body MB, and the other ends thereof may be connected to each other via a fastening means. In this case, the connecting means may be formed as magnetic fastening, Velcro fastening, pin fastening, and the like, but is not limited thereto. Further, the strap ST is not limited thereto, and may be integrally formed as a non-detachable band.

The main body MB may include the apparatus for estimating body temperature. A sensor 710, a processor, a display device, an output interface, a storage, and a communication interface may be mounted in the apparatus for estimating body temperature. However, depending on the size and shape of a form factor and the like, some of the display device, the storage, and the communication interface may be omitted.

A manipulator 720 may be formed on a side surface of the main body MB, as illustrated herein. The manipulator 720 may receive a user's command and may transmit the received command to the processor. In addition, the manipulator 720 may have a power button to turn on/off the wearable device 700.

The sensor 710 may include a photoplethysmogram (PPG) sensor. The PPG sensor may measure a PPG signal by emitting light onto an object and by detecting light scattered or reflected from the object.

The processor mounted in the main body MB may be electrically connected to various components including the sensor 710. For example, the processor may estimate cutaneous blood flow and energy metabolism based on the PPG signal measured by the PPG sensor, may estimate core body temperature based on the estimated cutaneous blood flow and energy metabolism, and may provide notification information on a risk of abnormal body temperature based on the estimated core body temperature. In addition, the processor may obtain cutaneous blood flow and energy metabolism based on a calibration PPG signal measured by the PPG sensor at a calibration time, may determine weights to be applied to the obtained cutaneous blood flow and energy metabolism, respectively, and may generate a core body temperature estimation model for estimating the core body temperature by linearly combining the weighted values.

A display may be provided on a front surface of the main body MB and may display various application screens, including body temperature information, time information, received message information, notification information on the risk of abnormal body temperature, and the like.

Referring to FIG. 8, the electronic device may be implemented as a mobile device 800 such as a smartphone.

The mobile device 800 may include a housing and a display panel. The housing may form an outer appearance of the mobile device 800. The housing has a first surface, on which a display panel and a cover glass may be disposed sequentially, and the display panel may be exposed to the outside through the cover glass. A sensor 810, a camera module and/or an infrared sensor, and the like may be disposed on a second surface of the housing.

For example, a plurality of sensors for obtaining data from a user may be disposed on a rear surface of the smartphone 800, and a fingerprint sensor disposed on the front surface thereof, a power button or a volume button disposed on a side surface thereof, a sensor disposed on other positions of the front and rear surfaces thereof, and the like may be provided to estimate the core body temperature.

The mobile device 800 may provide a user interface 820 via the display panel, to receive health profile information including a weight, a height, an age, and a temperature measurement site from a user input.

In addition, when a user transmits a request for estimating body temperature by executing an application and the like installed in the mobile device 800, the mobile device 800 may obtain data by using the sensor 810, and may estimate the core body temperature and may provide the estimated value and/or the notification information on the risk of abnormal body temperature to the user by using the processor in the mobile device 800.

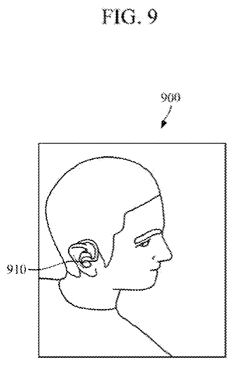

Referring to FIG. 9, the electronic device may be implemented as an ear-wearable device 900.

The ear-wearable device 900 may include a main body and an ear strap. A user may wear the ear-wearable device 900 by hanging the ear strap on the user's auricle. The ear strap may be omitted depending on the shape of ear-wearable device 900. The main body may be inserted into the external auditory meatus. A sensor 910 may be mounted in the main body. The ear-wearable device 900 may provide a user with a body temperature estimation result and/or the notification information on the risk of abnormal body temperature as sound, or may transmit the information to an external device, e.g., a mobile device, a tablet PC, a personal computer, etc., through a communication module provided in the main body.

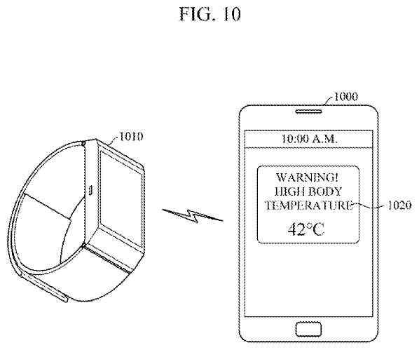

Referring to FIG. 10, the electronic device may be implemented as a combination of a wristwatch-type wearable device and a smartphone. For example, a processor for estimating core body temperature may be mounted in a main body of the mobile device 1000. Upon receiving a request for measuring body temperature, the processor of the mobile device 1000 may communicate with a communication interface, mounted in the main body of the wearable device 1010, to obtain data through the communication interface. Further, upon receiving data, such as a PPG signal and the like from the wearable device 1010, the processor may estimate core body temperature. For example, if the estimated core body temperature is in a range greater than or equal to a predetermined threshold range, the processor may output a notification message 1020 indicating a risk of abnormal body temperature, “WARNING!HIGH BODY TEMPERATURE,” along with the estimated core body temperature on a display of the smartphone 1000, as illustrated herein.

While not restricted thereto, an example embodiment can be embodied as computer-readable code on a computer-readable recording medium. The computer-readable recording medium is any data storage device that can store data that can be thereafter read by a computer system. Examples of the computer-readable recording medium include read-only memory (ROM), random-access memory (RAM), CD-ROMs, magnetic tapes, floppy disks, and optical data storage devices. The computer-readable recording medium can also be distributed over network-coupled computer systems so that the computer-readable code is stored and executed in a distributed fashion. Also, an example embodiment may be written as a computer program transmitted over a computer-readable transmission medium, such as a carrier wave, and received and implemented in general-use or special-purpose digital computers that execute the programs. Moreover, it is understood that in example embodiments, one or more units of the above-described apparatuses and devices can include circuitry, a processor, a microprocessor, etc., and may execute a computer program stored in a computer-readable medium.

The foregoing exemplary embodiments are merely exemplary and are not to be construed as limiting. The present teaching can be readily applied to other types of apparatuses. Also, the description of the exemplary embodiments is intended to be illustrative, and not to limit the scope of the claims, and many alternatives, modifications, and variations will be apparent to those skilled in the art.

What is claimed is:

1. An apparatus for estimating body temperature, the apparatus comprising:
a photoplethysmogram (PPG) sensor configured to measure a PPG signal by emitting light onto an object and by detecting light scattered or reflected from the object; and
a processor configured to estimate a cutaneous blood flow and an energy metabolism of the object based on the PPG signal, estimate a core body temperature of the object based on the cutaneous blood flow and the energy metabolism, and provide notification information on a risk of abnormal body temperature based on the core body temperature.

2. The apparatus of claim 1, wherein the processor is further configured to obtain a reference cutaneous blood flow and a reference energy metabolism based on a calibration PPG signal measured by the PPG sensor at a calibration time, determine weights to be applied to the reference cutaneous blood flow and the reference energy metabolism, respectively, and generate a core body temperature estimation model for estimating the core body temperature based on a weighted sum of the reference cutaneous blood flow and the reference energy metabolism.

3. The apparatus of claim 2, wherein the processor is further configured to determine a first weight for the cutaneous blood flow based on heat generated from an external heat source, and determine a second weight for the energy metabolism based on an internally generated heat.

4. The apparatus of claim 1, wherein the processor is configured to extract a feature from the PPG signal, and estimate the cutaneous blood flow based on the extracted feature by using a model that defines a correlation between the feature and the cutaneous blood flow.

5. The apparatus of claim 1, wherein the processor is further configured to obtain a heart rate from the PPG signal, and estimate the energy metabolism based on the heart rate.

6. The apparatus of claim 1, wherein the processor is further configured to estimate the core body temperature of the object by applying the cutaneous blood flow and the energy metabolism to a predetermined core body temperature estimation model.

7. The apparatus of claim 1, further comprising an output interface, wherein in response to the estimated core body temperature falling outside a predetermined threshold range, the processor is further configured to provide the notification information on the risk of abnormal body temperature through the output interface.

8. The apparatus of claim 1, wherein in response to the estimated core body temperature falling outside a predetermined temperature range, the processor is further configured to provide a text message or a voice message that recommends stopping outdoor activities.

9. The apparatus of claim 1, wherein the processor is further configured to estimate the core body temperature of the object based further on at least one of a user's motion and temperature while the PPG signal is measured.

10. A method of estimating body temperature, the method comprising:
measuring a photoplethysmogram (PPG) signal by emitting light onto an object and by detecting light scattered or reflected from the object;
estimating cutaneous blood flow and energy metabolism based on the PPG signal;
estimating core body temperature of the object based on the cutaneous blood flow and the energy metabolism; and
providing notification information on a risk of abnormal body temperature based on the estimated core body temperature.

11. The method of claim 10, further comprising obtaining a reference cutaneous blood flow and a reference energy metabolism based on a calibration PPG signal at a calibration time, determining weights to be applied to the reference cutaneous blood flow and the reference energy metabolism, respectively, and generating a core body temperature estimation model for estimating the core body temperature based on a weighted sum of the reference cutaneous blood flow and the reference energy metabolism.

12. The method of claim 11, wherein the generating of the core body temperature estimation model comprises determining a first weight for the cutaneous blood flow based on heat generated from an external heat source, and determining a second weight for the energy metabolism based on an internally generated heat.

13. The method of claim 10, wherein the estimating of the cutaneous blood flow comprises extracting a feature from the PPG signal, and estimating the cutaneous blood flow based on the extracted feature by using a model that defines a correlation between the feature and the cutaneous blood flow.

14. The method of claim 10, wherein the estimating of the energy metabolism comprises obtaining a heart rate from the PPG signal, and estimating the energy metabolism based on the heart rate.

15. The method of claim 10, wherein the estimating of the core body temperature of the object comprises estimating the core body temperature of the object by applying the cutaneous blood flow and the energy metabolism to a predetermined core body temperature estimation model.

16. The method of claim 10, wherein the providing of the notification information on the risk of abnormal body temperature comprises, in response to the estimated core body temperature falling outside a predetermined threshold range, providing the notification information on the risk of abnormal body temperature through an output interface.

17. The method of claim 10, further comprising, in response to the core body temperature falling outside a predetermined threshold range, providing a text message or a voice message that recommends stopping outdoor activities.

18. The method of claim 10, wherein the estimating of the core body temperature of the object comprises estimating the core body temperature of the object based further on at least one of a user's motion and temperature while the PPG signal is measured.

19. An electronic device comprising:
a display configured to provide a user interface to receive health profile information;
a PPG sensor configured to measure a PPG signal by emitting light onto a user and by detecting light scattered or reflected from the user; and
a processor configured to estimate a cutaneous blood flow and an energy metabolism of the user based on the PPG signal, estimate a core body temperature of the user based on the cutaneous blood flow and the energy metabolism, and provide notification information on a risk of abnormal body temperature based on the core body temperature, and the health profile information.

20. The electronic device of claim 19, wherein the health profile information comprises a weight, a height, and an age of the user, and one of a plurality of temperature measurement sites in a body of the user which is selected by the user.
